(19) United States
(12) Patent Application Publication
(10) Pub. No.: US 2023/0268053 A1
(43) Pub. Date: Aug. 24, 2023

(54) DIGITAL THERAPEUTIC PLATFORM FOR IMPROVING GUT HEALTH; METHOD AND SYSTEM INCUDING THE SAME

(71) Applicant: Bettermeal Inc., San Jose, CA (US)

(72) Inventor: Swathi Arulguppe Nagendra, San Jose, CA (US)

(21) Appl. No.: 18/143,033

(22) Filed: May 3, 2023

Related U.S. Application Data
(63) Continuation-in-part of application No. 18/105,190, filed on Feb. 2, 2023, now abandoned.
(60) Provisional application No. 63/306,597, filed on Feb. 4, 2022.

(51) Int. Cl. G16H 20/60; A61B 5/00; G16H 50/20; G16H 50/50

(52) U.S. Cl. CPC G16H 20/60; A61B 5/42; G16H 50/20; G16H 50/50

(57) ABSTRACT

Systems, methods, and apparatuses for analyzing markers of gut health, determining whether integrative or functional therapy is best, and incentivizing a prescribed regimen.

### RELATED APPLICATIONS

The present application is a continuation-in-part (CIP) of U.S. Pat. Application No. 18/105,190, filed Feb. 2, 2023, which claims the benefit of U.S. Provisional Pat. Application No. 63/306,597, filed Feb. 4, 2022, the contents of each of which, are incorporated by reference in their respective entireties for all purposes.

### FIELD OF THE DISCLOSURE

The present disclosure relates generally to gut health. In particular, but not by way of limitation, the present disclosure relates to systems, methods and apparatuses for analyzing markers of gut health, determining whether integrative or functional therapy is best, and then incentivizing a prescribed regimen.

### TECHNICAL PROBLEM

Traditional primary care tends to treat isolated symptoms regardless of any combination of symptoms. Functional and integrative nutrition are preventative approaches to healthcare largely focusing on discovering and addressing the root cause of symptoms through proper nutrition. More specifically, functional, and integrative nutrition are holistic approaches to diet, taking into consideration lifestyle factors that could affect food choices, such as activity levels, environment, or the presence of chronic disease. Standard nutrition focuses on the nutritional facts, such as a food or food group’s ability to promote or damage health (whether it’s “good” or “bad” for you). Functional and integrative nutrition looks at these facts, too, but in the context of an individual’s physiological makeup and how they live, such as how often they move, the quality of their relationships, and their stress level. Many approaches to functional and integrative nutrition are known in the art.

However, existing approaches to nutrition suffer from ins and outs of metabolism, micronutrients, hormones, and gut microbiology which are most salient factors. People do not have proven food-based cures to help reverse disease progression. There are biased recommendations from Nutritionists and Gut tests kits-based recommendations are not accurate. There are only disease management or general health recommendation mobile applications available today and there is therefore a need in the art for a digital therapeutic platform that provides advanced data driven precise nutritional solutions that addresses the root cause of the health issues which is gut health targeted towards specific conditions.

### SUMMARY OF EXAMPLE TECHNICAL SOLUTION

In view of the limitations of the traditional primary care and existing approaches to functional and integrative nutrition, the present disclosure provides a digital therapeutic platform that can offer advanced data-driven precise nutritional solutions for addressing the root cause of health issues, specifically targeting gut health, and focusing on specific conditions. The present disclosure further provides a method and system of using the digital therapeutic platform. The digital therapeutic platform may utilize a combination of advanced machine learning techniques, personalized biomarker data, and predictive modeling to deliver tailored functional nutrition plans that consider each individual’s unique physiological makeup, lifestyle factors, and disease progression.

In some examples, the digital therapeutic platform comprises: a sample collection module for obtaining biological material from a patient, such as blood and fecal samples; a biomarker identification module for identifying a plurality of biomarkers in the obtained samples; a data analysis module for analyzing the obtained samples using machine learning techniques, such as principal component analysis, heatmap, and Spearman correlation; a functional nutritional profile prediction module for predicting functional nutritional profiles for microbial communities using ensemble models, such as neural networks, Random Forests with multiple CART models, and Topological Network Analysis; an integration module for integrating the topological network of microbes with a predictive model, such as a Boolean potent model; an inflammation score prediction module for predicting an inflammation score using the predictive model; a p-value adjustment module for adjusting p-values for multiple comparisons; and a functional nutrition determination module for determining the functional nutrition required for improving the unique microbiome causing the disease progression based on the predicted inflammation score.

The digital therapeutic platform may also include a personalized nutrition plan tailoring module that uses the predicted functional nutritional profiles to develop customized nutrition plans for each patient, taking into account their unique physiological makeup, lifestyle factors, and disease progression. The platform may also comprise a monitoring module for tracking the patient’s inflammation score over time, thereby assessing the efficacy of the administered functional nutrition, and allowing for adjustments in the nutrition plan as needed.

By implementing the digital therapeutic platform according to the present disclosure, patients suffering from specific conditions can receive personalized, data-driven nutritional solutions that address the root cause of their health issues, particularly gut health, and help reverse disease progression. This platform overcomes the limitations of biased recommendations from nutritionists, inaccuracies in gut test kit-based recommendations, and the inadequacies of existing disease management or general health recommendation mobile applications.

### BRIEF DESCRIPTION OF THE DRAWINGS

FIG. 1A and FIG. 1B depict relative differences in microbiota in active functional and/or integrative treatment patients and those not using functional and/or integrative treatment.

FIG. 2 is a flowchart illustrating an example method of calculating gut score, selecting a functional or integrative nutritional treatment plan, and generating a personalized nutritional plan.

FIG. 3 is a flowchart depicting another method of calculating gut score, selecting a functional or integrative nutritional treatment plan, and generating a personalized nutritional plan.

FIG. 4 is a flowchart presenting yet another method of calculating gut score, selecting a functional or integrative nutritional treatment plan, and generating a personalized nutritional plan.

FIG. 5 is a flowchart illustrating a method of determining and presenting a personalized nutrition plan through a user interface.

FIG. 6 illustrates an example system for presenting a personalized nutritional plan to a user interface based on functional or integrative nutrition, taking into account user inputs such as food preferences, dietary behavior, allergies, and other factors.

FIG. 7 shows an example block diagram of physical components that can be used to realize a user computing device or remote server(s) for implementing the methods and systems disclosed in previous figures.

FIG. 8 depicts an example method for determining a patient’s inflammation index, selecting a nutritional treatment plan, and generating a personalized meal plan based on biomarker sequences and bioactive compounds in food.

FIG. 9 is a flowchart illustrating an example method for reducing inflammation in a patient.

### DETAILED DESCRIPTION

The word “exemplary” is used herein to mean “serving as an example, instance, or illustration.” Any embodiment described herein as “exemplary” is not necessarily to be construed as preferred or advantageous over other embodiments.

Preliminary note: the flowcharts and block diagrams in the following Figures illustrate the architecture, functionality, and operation of possible implementations of systems, methods and computer program products according to various embodiments of the present disclosure. In this regard, some blocks in these flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer, or section. Thus, a first element, component, region, layer, or section discussed below could be termed a second element, component, region, layer, or section without departing from the teachings of the present disclosure.

Spatially relative terms, such as “beneath,” “below,” “lower,” “under,” “above,” “upper,” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items and may be abbreviated as “/”.

It will be understood that when an element or layer is referred to as being “on,” “connected to,” “coupled to,” or “adj acent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adj acent to” another element or layer, there are no intervening elements or layers present.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

“Principal component analysis” is a statistical technique that reduces the dimensionality of a dataset while retaining key information.

A “heatmap” is a graphical representation of data using colors to represent numerical values, typically used for visualizing complex datasets.

“Spearman correlation” is a nonparametric measure of statistical dependence between two variables, assessing the degree of association between the ranked variables.

An “ensemble model” refers to a combination of multiple machine learning models used together to improve the overall predictive performance. Ensemble models can help in achieving more accurate and robust results by leveraging the strengths of each individual model while compensating for their weaknesses.

A “topological network of microbes” is a graphical representation of the interconnections and relationships among different types of microbiomes. This network analysis helps in identifying associations between beneficial and harmful bacteria and understanding interactions within the microbial community.

“A Boolean potent model” is a type of predictive model that uses Boolean logic to determine the presence or absence of important bioactive compounds in food. It helps in understanding the potential impact of these compounds on health and inflammation.

The “false nutritional discovery rate” refers to the probability of incorrectly identifying bioactive compounds in food types. This metric is used to reanalyze and align the correct bioactives, ensuring accurate nutritional information and reducing the chances of false discoveries.

“Random Forests” are an ensemble learning method that constructs multiple decision trees (“CART” models) and combines their predictions. Each “CART” (Classification and Regression Trees) model is responsible for analyzing bacterial sequences, while the topological network analysis reveals the connections between the gut microbiome and biomarkers, aiding in the development of effective nutritional solutions.

Analysis of Variance (“ANOVA”) tests are a collection of statistical models used to analyze the differences among group means by comparing the variation between groups with the variation within groups. In the context of this disclosure, ANOVA tests help to establish the statistical connection between the microbiome and bioactive compounds, providing a solid foundation for the development of personalized nutrition plans.

This disclosure presents systems and methods for determining whether to apply functional or integrative solutions based on a novel algorithm that assumes that gut health is a key indicator of the correct therapeutic path. Gut health is quantified by a “gut score” that is at least based on biomarkers, fecal analysis, user symptoms, nutrition, overall health behavior, and environmental factors such as relationship status and other stress inducers. Unique approaches to these two therapies are then implemented to restore gut health based on the novel assumption that gut health is more important to overall well-being than is recognized in the literature. Goals and incentive-based therapies can also be used to encourage user adherence to suggested therapies. For instance, t-shirts, hats, bumper stickers, athletic wear, gift cards, audio books, and non-fungible tokens (NFTs), to name a few, can be used as rewards to incentive user adherence to therapies.

### Functional Approach to Nutrition

The functional nutrition used in this disclosure addresses the root cause of health issues through proper cellular nourishment. Bodies react to imbalances therein, and the disclosed systems and methods provide functional therapies to optimize a path forward for each individual. Imbalances can include, but are not limited to, inflammation, hormonal imbalance, digestive and microbial factors, and levels of toxicity. These imbalances can lead to chronic diseases such as PCOS, Obesity, Diabetes, irritable Bowel Disease, Thyroid issues, Celiac Diseases, and other Auto-immune diseases. The functional therapies disclosed herein take into account gut health, biomarkers (e.g., Complete Blood Count-“CBC”), stool tests, eating habits, symptoms and lifestyle, and result in nutrition plans aimed at identifying underlying factors of il-health as well as repair imbalances with proper nutrients and the biochemical and metabolic benefits that result therefrom. Therapies are formulated from the perspective that health and disease exist on a continuance.

### Integrative Approach to Nutrition

The disclosed systems and methods can provide integrative nutrition, or a type of dietetics that optimizes physiological functioning of cells. When cells are nourished with appropriate foods, the root causes of multiple symptoms can be addressed. These systems and methods also connect system imbalances with root causes of ill-health and help reverse disease progression, and can include, but are not limited to, macronutrients, micronutrients, energy expenditure, and food labels. The disclosed integrative approach explores solutions to gut health based on, among other things, biomarker sequencing, without dependency on external test kits.

More specifically, the disclosed integrative approach is based on discovered connections between specific foods and whole-food supplements and depression, irritability, emotional eating, and behavior. In other words, food selection is based at least in part on emotional state. Such food selection largely comprises fruits, vegetables, and healthy fats that can alleviate depression and decrease irritability. The disclosed integrative therapy also includes fixing food cravings and addictions by identifying those cravings and educating users as to how best to respond to cravings. This disclosure also deconstructs cravings by evaluating the type of food, the timing of the craving, the brain activity occurring during cravings, and environmental events surrounding the craving. Deconstruction also looks at chemicals associated with cravings.

### Biomarker Sequencing

Based on empirical analysis of 500,049 patient records of those suffering from chronic health issues, including 220,465 Diabetic patients, 102,000 PCOS patients, 63,000 Thyroid patients, 100,030 Celiac patients, and 14,554 patients with Irritable Bowel Syndrome, complete blood count results and stool tests were analyzed, and specific biomarkers tabulated. The biomarkers analyzed were Neutrophils, Eosinophils, Monocytes, Lymphocytes, BUN ratio, A/G Ratio, ALT, Total carotenoids, products of protein fermentation, fecal fat, and fat-soluble vitamins. Note that these patients were actively consuming prescription medicines and Vitamins before any functional or integrative therapies were applied. Alanine aminotransferase and aspartate aminotransferase are one of the dependable markers, giving the survey metabolic syndrome analysis with gut permeability. Also, tests for H. Pylori were performed and nearly 52% of the patient pool having H. Pylori had elevated ALT/AST levels. CBC and Fecal primers were used to step up a large shred and an example sequence for a Diabetes patient was: Forward Basal Index F1 (AA1NEMLBWFpC) and Reverse Basal Index F6 (MLBWFpCA1AEN). With this sequence the microbial variance was high and optimal 25 unique microbial markers were identified. The analysis was re-captured for patients with Thyroid, Celiac, PCOS and IBD, with more than 35 unique biomarkers identified, which clearly verified the deprived balance of good and bad bacteria along with other pathogens that had been compromising the gut barrier. The tabulated sequence reads down to the microbiota level.

### Data Analysis

Cut-off values for biomarker sequences were calculated from blood samples, stool samples, and a nutritional database that includes healthy controls. The sequence cut-off values were used to identify positive samples with at least 6 unique inflammatory microbiomes in the sequence. Then the sequence was analyzed using principal component analysis (PCA), heatmap, and spearman correlation. Functional nutritional profiles for microbial communities were predicted using ensemble models using generative adversarial neural networks (GANS), Random Forests, having multiple CART models and Topological Network Analysis. The topological network of microbes was integrated with a Boolean potent model which was modeled from a time series genomic data. The model also predicted the inflammation score, and ANOVA tests were performed with p-values being adjusted for multiple comparisons using the false nutritional discovery rate. The value p < 0.05 was considered statistically significant. The complex models were tied together to predict the functional nutrition suggested for improving the (poor) unique microbiome which was the cause of patient disease progression.

### Tests And Treatment

After 1, 3 and 6 months of analyzing without any formal treatment, the patients were referred to their specialists. The doctors generally related that the patients felt “82% healed” but were seeking a 100% recovery. The patients were tested again after 6 months of functional and/or integrative treatments and tests were performed to observe any bacteria cross feeding. The Bacteria cross feeding was checked from the fecal samples looking for fumarate, succinate, and lactate, and these were detected at low levels < 10% in the observed feces adding an indicator of success to the experiments. The figure shows the relative increase in the omics mix in the active meal plan patients as compared to patients without functional and/or integrative treatments after 6 months of analysis.

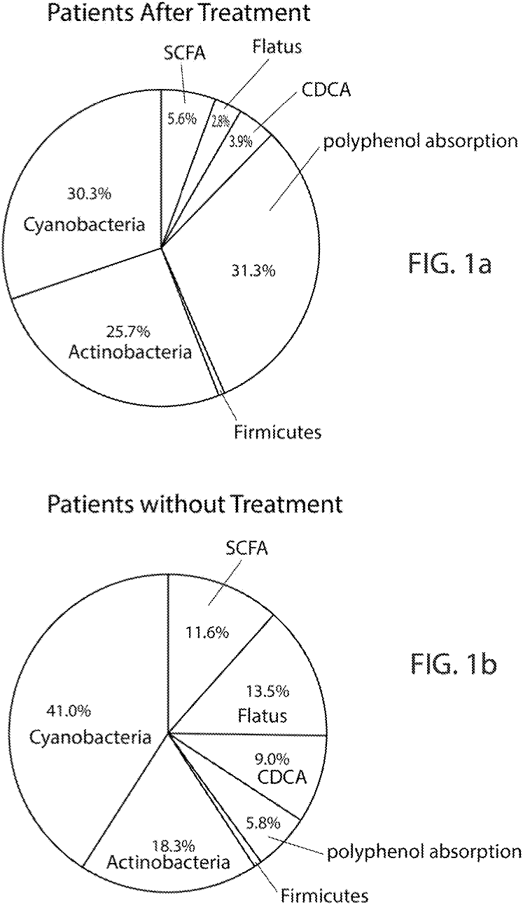

FIGS. 1A and 1B show relative differences in microbiota in active functional and/or integrative treatment patients and those not using functional and/or integrative treatment.

With the patients exhibiting good omics strength with the high-functioning nutrition plan, the suspected possible inflammation was reduced dramatically by 62%. The basis for treatment was a researched and documented nutritional therapy protocol and the goal was to reverse the patient’s conditions with strict nutritional changes.

### Results

Table 1 shows relative differences in microbiota in active functional/integrative treatment patients and non-functional/integrative treatment patients.

TABLE 1

Log2 Fold Change

| Phylogeny | K Fold Change Meal Plan vs Non meal plan | p-value |
|---|---|---|
| Actinobacteria | 2.103 | < 0.001 |
| Firmicutes | 0.23 | < 0.01 |
| Cyanobacteria | -0.446 | < 0.05 |

The essence of biochemical capability within the omics extends its flexibility to act on a wide range of dietary granules. Nutrients based metabolism is a major activation function of the microbiota along with the biochemical pathways. The omics also have substantial ability to metabolize phytochemicals, particularly SCFAs, CDCAs, Flatus and polyphenols, by diverse, well classified nutrient pathways. There is strong evidence in the study that shows individual differences in biochemical change of omics on health due to different sets of nutrition. The partake and usage of omic-based digestion was also studied by considering other members of the gut microbiota and observing the integration by nutrition sets that resulted in omics which were non-inflammatory. From this clinical study it was inferred that 39% of PCOS patients reported no symptoms after 6 months. 46% Thyroid patients reported very few symptoms and 12% among them could conceive naturally. 51% Diabetes patients had a major dip in glucose and stayed consistent after 6 months.

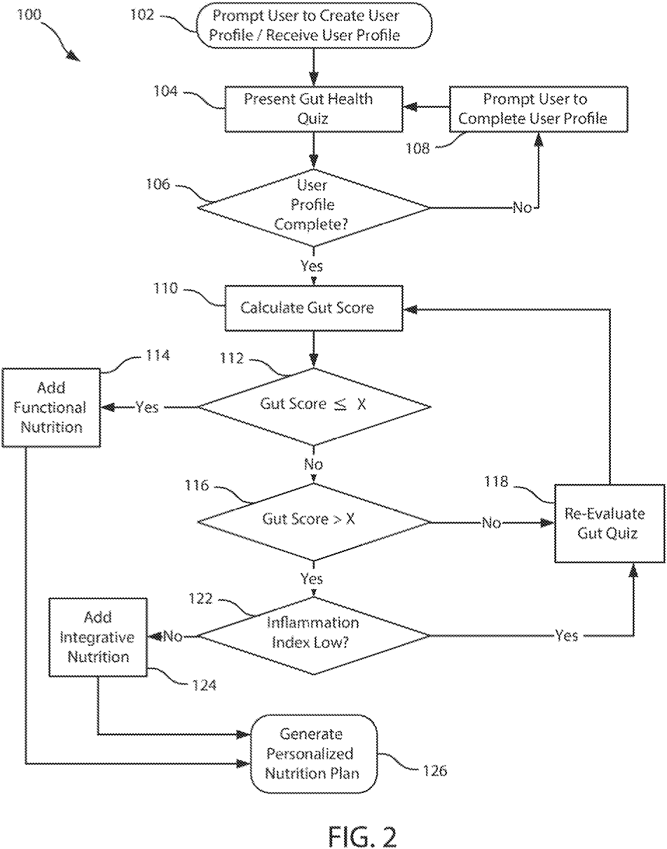

FIG. 2 illustrates a method of calculating gut score, selecting a functional or integrative nutritional treatment plan, and generating a personalized nutritional plan based thereon. The method 100 starts by prompting a user to create a user profile or receiving a user profile (Block 102). The method 100 then presents a gut health quiz (Block 104), for instance in an app or web browser. Answers to the quiz are received/collected and the method 100 determines if the user profile is complete (Decision 106). If not, then the method 100 prompts the user to create/complete their profile (Block 108). Once the profile is deemed complete, a gut score is calculated based on the quiz and other factors (Block 110). The gut score is then used to determine whether functional or integrative nutrition will be used to generate a personalized nutrition plan. In particular, if the gut score is less than or equal to a threshold (e.g., 40) (Decision 112), then the method 100 generates the personalized nutrition plan (Block 126) based on functional nutrition (Block 114). If the gut score is greater than this threshold (e.g., greater than 40) (Decision 116), the method 100 determines if the user’s inflammation index is low (Decision 122). As used in this context, “low” inflammation is defined as when the user’s inflammation index does not exceed a predetermined inflammation threshold. If the inflammation is not low, then integrative nutrition (Block 124) is used to generate the personalized nutrition plan (Block 126). If the inflammation index is low (Decision 122 = Yes), then the method 100 re-evaluates the gut health quiz (Block 118) and recalculates the gut score (Block 110). In the rare event that the gut score does not meet the conditions of Decision 112 or 116, re-evaluation of the gut quiz (Block 118) and a recalculation thereof (Block 110) can occur.

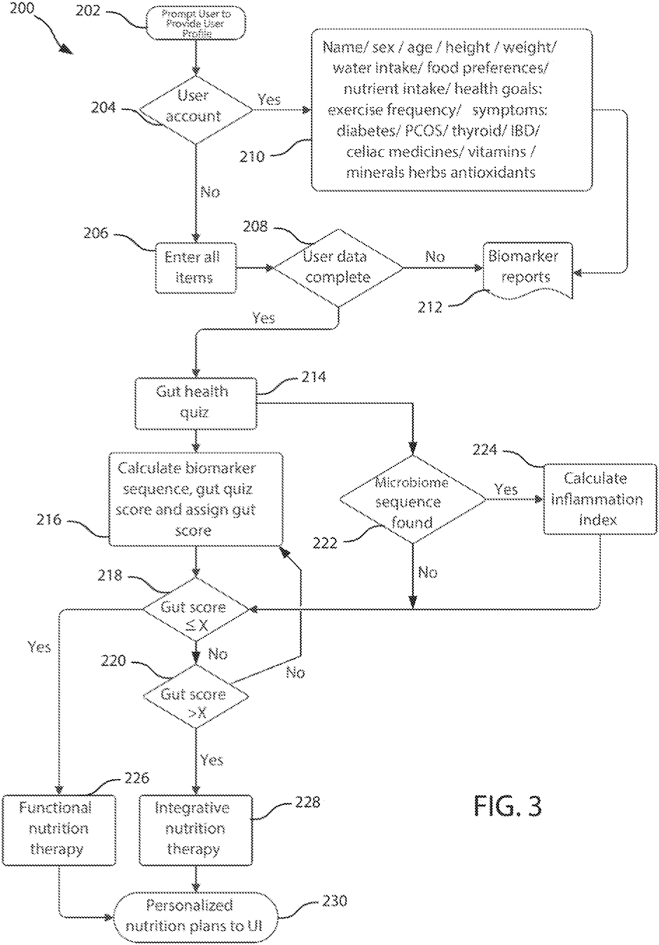

FIG. 3 illustrates another method of calculating gut score, selecting a functional or integrative nutritional treatment plan, and generating a personalized nutritional plan based thereon. The method 200 starts by prompting a user to create a user profile or receiving a user profile (Block 202). The method 200 then determines if a user account exits (Decision 204) and if not, the user is prompted to enter missing items (Block 206). If a user account does exist (Decision 204 = Yes), then the method 200 generates a biomarkers report(s) 212. When complete, the account can include information such as, but not limited to, name, sex, age, height, weight, daily water consumption, food preferences, daily nutrient intake, health goals, exercise frequency, symptoms, and any known health issues such as diabetes, PCOS, thyroid, IBD, and currently-used non-food intake such as celiac medicines, vitamins, minerals, herbs, and antioxidants. When the user data is complete (Decision 208 = Yes), a gut health quiz is presented to the user (Block 214), for instance in an app or web browser. Answers to the quiz are received/collected and method 200 calculates the bio marker sequence, gut quiz score, and from these calculates a gut score (Block 216). The gut score is then used to determine whether functional or integrative nutrition will be used to generate a personalized nutrition plan. In particular, if the gut score is less than or equal to a threshold X (e.g., 40), then functional nutrition (Block 226) can be used to generate a personalized nutrition plan that is presented via a user interface (block 230). If the gut score is greater than the threshold X, then integrative nutrition (Block 228) can be used to generate the personalized nutrition plan and presented on the user interface (Block 230). At the same time, if a microbe sequence is found in the results of the gut quiz (Decision 222), then the method 200 calculates an inflammation index (Block 224). Whether a microbe sequence is found or not, the method 200 continues by looking at the gut score (Decision 218) as described earlier.

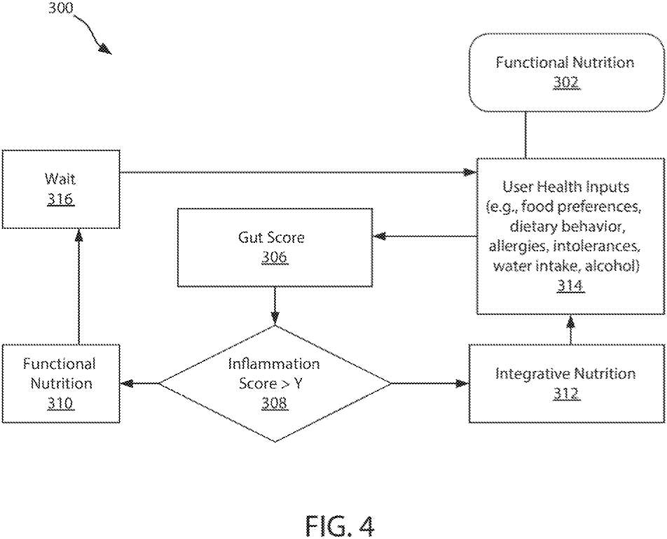

FIG. 4 illustrates yet another method of calculating gut score, selecting a functional or integrative nutritional treatment plan, and generating a personalized nutritional plan based thereon. The method 300 defaults to application of functional nutrition 302 to a personalized nutrition plan displayed on a user interface. User health inputs are then received via the user interface or other inputs (e.g., a keyboard or voice input) (Block 314). These can include, but are not limited to, food preferences, dietary behavior, allergies, intolerances, water intake, and alcohol consumption. The user interface then presents a gut score quiz and user inputs are converted to a gut score (Block 306). The method 300 also analyzes inflammation to produce an inflammation score and compare this to a threshold Y (e.g., 50). If the inflammation index is greater than a threshold Y (Decision 308 = Yes), then the user interface presents a personalized nutrition plan based on functional nutrition (Block 310). On the other hand, if the inflammation index is less than the threshold Y (Decision 308 = No), then the user interface presents a personalized nutrition plan based on integrative nutrition (Block 312).

If integrative nutrition (Block 312) is implemented, then method 300 again monitors/receives user inputs (Block 314) and again determines a gut score (Block 306), possibly from a new gut quiz. If functional nutrition (Block 310) is implemented, then method 300 waits for some period of time, such as two, three, or five months, and monitors/receives user inputs during this time (Block 314). After the time has elapsed, another gut score is determined (Block 306). This process continues to cycle through user inputs, gut scores, inflammation comparisons to the threshold, Y, and application of functional or integrative nutrition. In this way, the software can periodically or continually update its analysis to determine whether functional or integrative nutrition is most beneficial at any given time-sometimes even jumping between the two.

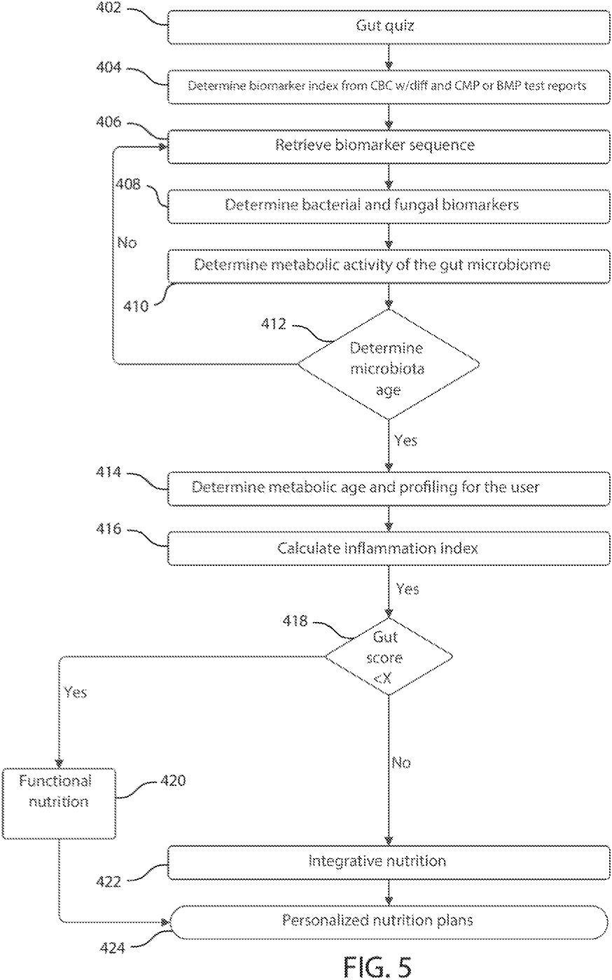

FIG. 5 illustrates a method of determining and presenting a personalized nutrition plan through a user interface. The method 400 starts with a gut quiz (Block 402) that can be presented via a user device such as a laptop, smartphone, tablet, etc. Questions can be multiple choice, open ended, or answerable via voice input, to name a few non-limiting examples. The results of the gut quiz can be used to determine a biomarker index from its CBC w/diff and CMP or BMP test reports (Block 404) and the method 400 can retrieve the biomarker sequence (Block 406). The method 400 can from this, determine bacterial and fungal biomarkers (Block 408) and determine metabolic activity of the gut microbiome (Block 410). The method 400 can then determine a microbiota age, and if the age is below a threshold (Decision 412 = No), then the method 400 returns to retrieving biomarker sequence (Block 406). Once the microbiota age can be determined (Decision 412 = Yes), the method can determine a metabolic age and profile for the user (Block 414) and calculate an inflammation index (Block 416). With this index and the metabolic age of the user, the method 400 determines a gut score, and if that score is below a threshold X, then the method 400 applies functional nutrition (Block 420) in presenting a personalized nutrition plan via the user interface (Block 424). If the gut score is not below the threshold X (Decision 418 = No), then the method 400 applies integrative nutrition (Block 422) in presenting a personalized nutrition plan via the user interface (Block 424).

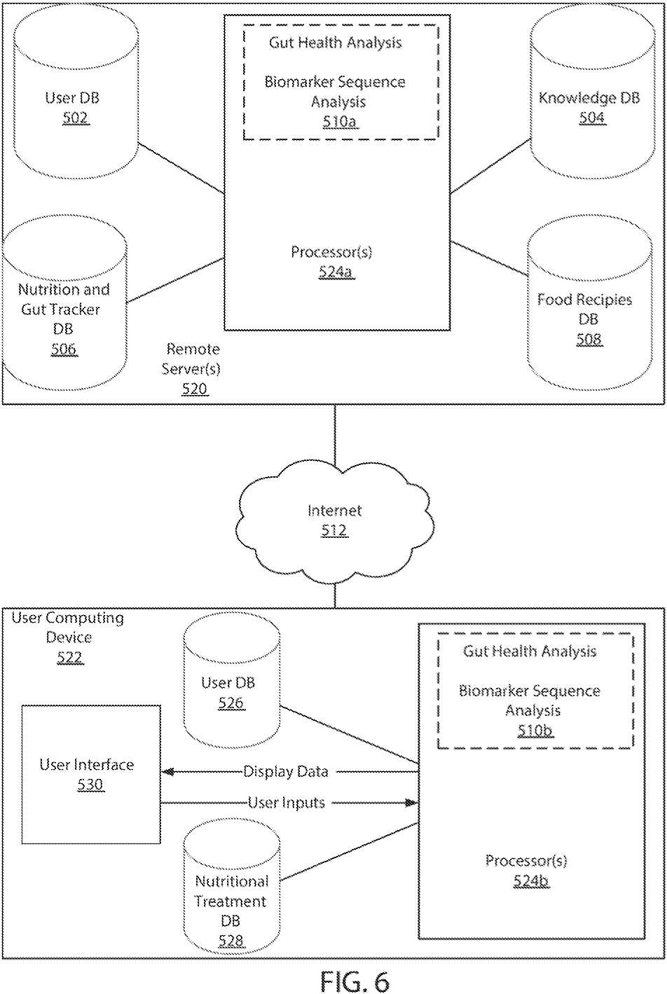

FIG. 6 illustrates a system for presenting a nutritional plan to a user interface. Ultimately, the plan is based on functional or integrative nutrition, whichever is best suited for a user at a given time. User inputs, such as food preferences, dietary behavior, allergies, intolerances, water intake, and alcohol consumption, to name a few, can be used to periodically or continually evaluate a user and select a functional or integrative approach for the nutritional plan. The system can largely include one or more remote server(s) 520 and at least one user computing device 522, such as a mobile phone, tablet, or laptop computer, to name a few non-limiting examples. These systems can be in digital communication via the Internet 512, including one or more wired and/or wireless connections. The use computing device 522 can include a user interface 530 configured to present questions to a user such as amount of water intake per day, alcohol consumption, dietary intolerances, allergies, food preferences, etc. These inputs can be received via one or more user input means, which may or may not be part of the user interface 530, such as a keyboard, touchscreen, and microphone. One or more processor(s) 524b can generate the questions and pass them to the user interface 530 as display data. Responses to the questions can arrive at the processor(s) 524b as user inputs, and then be used to populate a user profile that is stored on a user database 526 on the user computer device 522, although this user database 526 can also be stored or backed up on the one or more remote servers 520 (e.g., 502). This initial user profile can be used to generate a personalized nutrition plan based on functional nutrition. For instance, an optional gut health analysis 510a on the one or more remote servers 520 or an optional gut health analysis 510b on the user computing device 522 may perform this analysis. The processor(s) 524b can then generate a gut quiz and present this to the user via the user interface 530. The user’s responses can be returned to the processor(s) 524b and analyzed. For instance, biomarker sequence analysis 510b can be carried out if a biomarker sequence is available. Alternatively, the processor(s) 524a and the optional biomarker sequence analysis 510a on the one or more remote server(s) 520 can analyze the gut quiz responses. Depending on the gut score, the processor(s) 524b or 524a can use functional or integrative nutrition to develop a personalized nutrition plan for presentation to the user via the user interface 530. Scores and resulting plans can be stored locally, for instance, in the user DB 526 or remotely, for instance in the user DB 504.

The nutritional treatment DB 528 is a rapidly evolving database that offers unparalleled predictions of progress based on the user health history and biomarker sequences. In this DB 528, the system provides an overview of the main functional and integrative nutrition and identifies gaps to address specific health concerns. A deep learning algorithm helps better understand and predict the complex interactions between nutrition-related cures, particularly when large amounts of data is integrated. The DB 528 can also include image recognition criteria that improves dietary assessment by increasing efficiency and addressing systematic errors associated with self-reported calculations of dietary intakes. Finally, the DB 528 extracts, structures, and analyzes large amounts of data from patient portal platforms to better understand health history, dietary behaviors and perceptions.

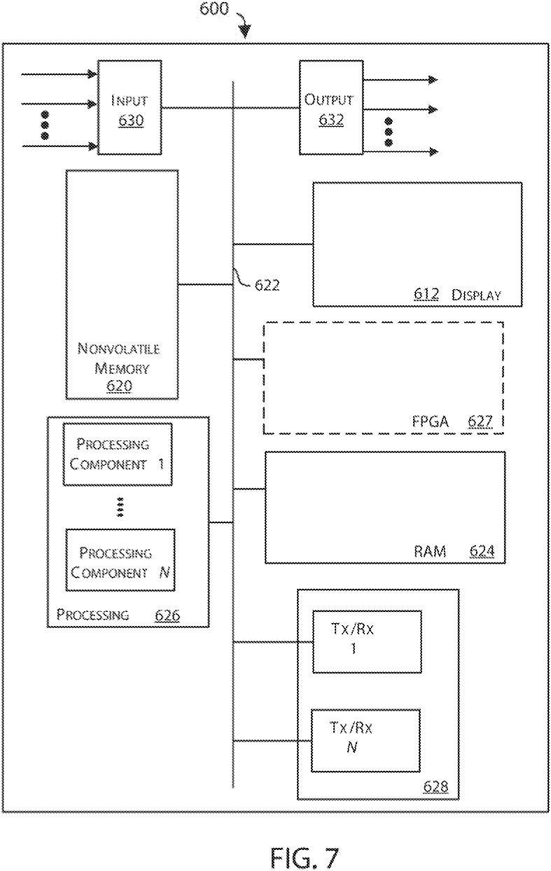

The methods described in connection with the embodiments disclosed herein may be embodied directly in hardware, in processor-executable code encoded in a non-transitory tangible processor readable storage medium, or in a combination of the two. Referring to FIG. 6 for example, shown is a block diagram depicting physical components that may be utilized to realize the user computing device 522 or the remote server(s) 520 (and the device for carrying out the methods disclosed herein generally) according to an exemplary embodiment. As shown, in this embodiment a display portion 612 and nonvolatile memory 620 are coupled to a bus 622 that is also coupled to random access memory (“RAM”) 624, a processing portion (which includes N processing components) 626, an optional field programmable gate array (FPGA) 627, and a transceiver component 628 that includes N transceivers. Although the components depicted in FIG. 7 represent physical components, FIG. 7 is not intended to be a detailed hardware diagram; thus, many of the components depicted in FIG. 7 may be realized by common constructs or distributed among additional physical components. Moreover, it is contemplated that other existing and yet-to-be developed physical components and architectures may be utilized to implement the functional components described with reference to FIG. 7.

This display portion 612 generally operates to provide a user interface for a user, and in several implementations, the display is realized by a touchscreen display. In general, the nonvolatile memory 620 is non-transitory memory that functions to store (e.g., persistently store) data and processor-executable code (including executable code that is associated with effectuating the methods described herein). In some embodiments for example, the nonvolatile memory 620 includes bootloader code, operating system code, file system code, and non-transitory processor-executable code to facilitate the execution of a method described with reference to FIGS. 2-5 described further herein.

In many implementations, the nonvolatile memory 620 is realized by flash memory (e.g., NAND or ONENAND memory), but it is contemplated that other memory types may be utilized as well. Although it may be possible to execute the code from the nonvolatile memory 620, the executable code in the nonvolatile memory is typically loaded into RAM 624 and executed by one or more of the N processing components in the processing portion 626.

The N processing components in connection with RAM 624 generally operate to execute the instructions stored in nonvolatile memory 620 to enable a method of presenting a personalized nutrition plan via a user interface that is based on either functional or integrative nutrition. For example, non-transitory, processor-executable code to effectuate the methods described with reference to FIGS. 2-6 may be persistently stored in nonvolatile memory 620 and executed by the N processing components in connection with RAM 624. As one of ordinarily skill in the art will appreciate, the processing portion 626 may include a video processor, digital signal processor (DSP), micro-controller, graphics processing unit (GPU), or other hardware processing components or combinations of hardware and software processing components (e.g., an FPGA or an FPGA including digital logic processing portions).

In addition, or in the alternative, the processing portion 626 may be configured to effectuate one or more aspects of the methodologies described herein (e.g., the method described with reference to FIGS. 2-6). For example, non-transitory processor-readable instructions may be stored in the nonvolatile memory 620 or in RAM 624 and when executed on the processing portion 626, cause the processing portion 626 to perform a method to present a personalized nutrition plan via a user interface that is based on either functional or integrative nutrition. Alternatively, non-transitory FPGA-configuration-instructions may be persistently stored in nonvolatile memory 620 and accessed by the processing portion 626 (e.g., during boot up) to configure the hardware-configurable portions of the processing portion 626 to effectuate the functions of the user computing device 522 or the remote server(s) 520.

The input component 630 operates to receive signals (e.g., the gut quiz answers) that are indicative of one or more aspects of the user’s gut health. The signals received at the input component may include, for example, keystrokes from the user computing device 522, audio answers to gut quiz questions, or radio selections on a touchscreen user interface of the user computer device 522. The output component generally operates to provide one or more analog or digital signals to effectuate an operational aspect of the user computing device 522 or the remote server(s) 520. For example, the output portion 632 may provide display data to the user interface 530 described with reference to FIG. 6.

The depicted transceiver component 628 includes N transceiver chains, which may be used for communicating with external devices via wireless or wireline networks. Each of the N transceiver chains may represent a transceiver associated with a particular communication scheme (e.g., WiFi, Ethernet, Profibus, etc.).

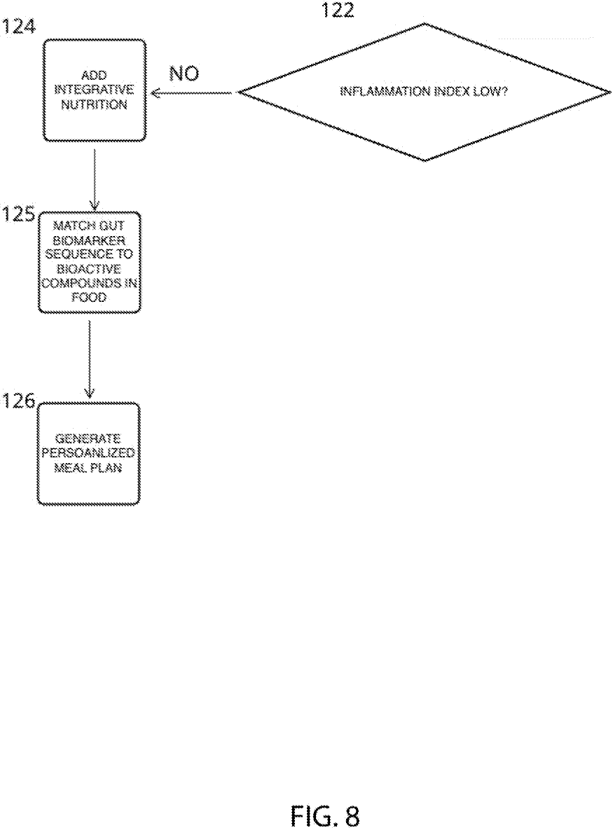

FIG. 8 depicts a further non-limiting example according to the present disclosure. In some examples, the steps of FIG. 8 correspond to the steps of FIG. 2, as shown above. However, this is not necessarily the case, and one or all of the steps of FIG. 8 may or may not be associated with at least one other embodiment of the present disclosure. As shown in FIG. 8, a method, system, or apparatus may include the following steps: determining whether a patient’s inflammation index is low 122; if no: adding integrative nutrition 124; matching a gut biomarker sequence to bioactive compounds in food 126; and generating a personalized meal plan 128. In some examples, existing lab results from the patient portal (CBC/w diff, CMP or BMP) or pdfs are included, and no new labs are required. In certain implementations, artificial intelligence, or machine learning technology (“AI/ML”) may be used to capture ratios of specific states of inflammation. AI/ML may also be used to connect bioactive compounds in food that are suitable to a patient’s gut, thereby providing a functional nutritional treatment that is suitable for the patient. In some examples, the AI/ML may predict a unique biomarker sequence and hidden food compounds that are bioactive and match sequence with the compounds in a way such that directly impacts at least one specific health condition of the patient. In some non-limiting examples, the specific health condition may be a health condition associated with the microbiome.

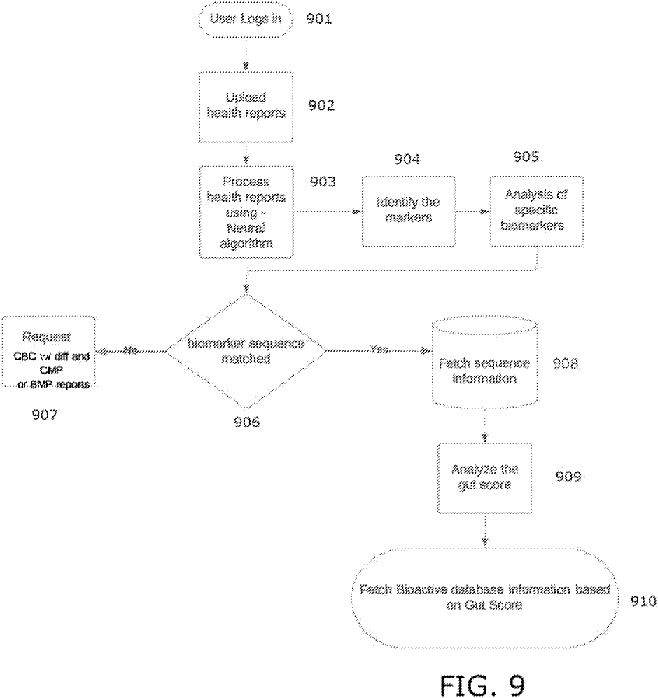

FIG. 9 is a flowchart illustrating a non-limiting example of a method for reducing inflammation in a patient. The patient may suffer from one or more diseases, such as, but not limited to, Thyroid, Celiac, Type 2 Diabetes, or PCOS, according to the present disclosure. The method may be implemented in a system or apparatus, and the steps of FIG. 9 may correspond to the steps of FIG. 2, as shown above. However, this is not necessarily the case, and one or all of the steps of FIG. 9 may or may not be associated with at least one other embodiment of the present disclosure.

Referring now to FIG. 9, at step 901, a user can log in to the system or platform. At step 902, the user can upload health reports, which may include existing lab results from the patient portal, such as CBC with differential, CMP, or BMP, or PDF files of lab results, and no new labs may be required.

At step 903, the health reports can be processed using a neural algorithm, which may include ensemble models, predictive models, multiple CART models, and Topological Network Analysis, among other machine learning techniques. In some implementations, the algorithm may also utilize principal component analysis, heatmap, Spearman correlation, false nutritional discovery rate, Random Forests, and ANOVA tests.

At step 904, a plurality of biomarkers in the obtained sample may be identified. Step 905 would then involve the analysis of specific biomarkers. At step 906, the system may determine whether the biomarker sequence is matched. If the biomarker sequence is matched, the system, may, in some embodiments, request CBC w/diff and CMP or BMP reports at step 907.

If the biomarker sequence is matched, the method proceeds to step 908, where sequence information is fetched. At step 909, the gut score is analyzed, which may involve the integration of the topological network of microbes with a predictive model and adjusting p-values for multiple comparisons.

At step 910, bioactive database information is fetched based on the analyzed gut score. The method may utilize artificial intelligence or machine learning technology to predict functional nutritional profiles for microbial communities and to connect bioactive compounds in food suitable for the patient’s gut, thereby providing a functional nutritional treatment that is suitable for the patient.

In some examples, the method may further predict a unique biomarker sequence and hidden food compounds that are bioactive and match the sequence with the compounds in a way that directly impacts at least one specific health condition of the patient. In some non-limiting examples, the specific health condition may be a health condition associated with the microbiome.

Some portions are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is a self-consistent sequence of operations or similar processing leading to a desired result. In this context, operations or processing involves physical manipulation of physical quantities. Typically, although not necessarily, such quantities may take the form of electrical or magnetic signals capable of being stored, transferred, combined, compared, or otherwise manipulated. It has proven convenient at times, principally for reasons of common usage, to refer to such signals as bits, data, values, elements, symbols, characters, terms, numbers, numerals, or the like. It should be understood, however, that all of these and similar terms are to be associated with appropriate physical quantities and are merely convenient labels. Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

As will be appreciated by one skilled in the art, aspects of the present disclosure may be embodied as a system, method, or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. A method for reducing inflammation in a patient, the method comprising:
obtaining a sample of the patient’s biological material;
identifying a plurality of biomarkers in the sample;
analyzing the obtained sample using at least one machine learning technique;
predicting a functional nutritional profile for microbial communities using at least one ensemble model;
integrating a topological network of microbes with at least one predictive model;
predicting an inflammation score using the at least one predictive model;
determining the functional nutrition profile required to improve a unique microbiome causing a disease progression based on the predicted inflammation score; and
administering the determined functional nutrition to the patient thereby reducing inflammation.

2. The method of claim 1, wherein the sample of the patient’s biological material comprises blood, fecal material, or any combination thereof.

3. The method of claim 1, wherein the at least one machine learning technique comprises principal component analysis, heatmap, Spearman correlation, or any combination thereof.

4. The method of claim 1, wherein the at least one ensemble model comprises at least one neural network, at least one Random Forest with multiple CART models, Topological Network Analysis, or any combination thereof.

5. The method of claim 1, wherein the at least one predictive model comprises a Boolean potent model.

6. The method of claim 1, wherein the functional nutritional profiles are predicted for specific microbial communities associated with the patient’s disease.

7. The method of claim 1, wherein the predicted functional nutritional profile is used to tailor a personalized nutrition plan for the patient.

8. The method of claim 1, further comprising monitoring the patient’s inflammation score over time to assess an efficacy of the administered functional nutrition.

9. The method of claim 1, where the patient has at least one of: Thyroid, Celiac, Type 2 Diabetes, PCOS, or any combination thereof.

10. A system for reducing inflammation in a patient:
a sample collection module configured to receive a patient’s biological material;
a biomarker identification module for configured to identify a plurality of biomarkers in the obtained sample;
a data analysis module configured to analyze the obtained sample using at least one machine learning technique;
a functional nutritional profile prediction module configured to predict functional nutritional profiles for microbial communities using at least one ensemble model;
an integration module configured to integrate a topological network of microbes with at least one predictive model;
an inflammation score prediction module configured to predict an inflammation score using the at least one predictive model;
a p-value adjustment module configured to adjust p-values for multiple comparisons;
a functional nutrition determination module configured to determine the functional nutrition required for improving a unique microbiome causing a disease progression based on the predicted inflammation score; and
a functional nutrition administration module configured to administer the determined functional nutrition to the patient, thereby reducing inflammation.

11. The system of claim 10, wherein the sample of the patient’s biological material comprises, blood, fecal material, or any combination thereof.

12. The system of claim 10, wherein the at least one machine learning technique includes principal component analysis, heatmap, Spearman correlation, or any combination thereof.

13. The system of claim 10, wherein the at least one ensemble model comprises at least one neural network, at least one Random Forest with multiple CART models, Topological Network Analysis, or any combination thereof.

14. The system of claim 10, wherein the at least one predictive model comprises a Boolean potent model.

15. The system of claim 10, wherein the functional nutritional profile is predicted for specific microbial communities associated with the patient’s disease.

16. The system of claim 10, wherein the predicted functional nutritional profiles are used to tailor a personalized nutrition plan for the patient.

17. The system of claim 10, further comprising a monitoring module for tracking the patient’s inflammation score over time to assess an efficacy of the administered functional nutrition.

18. The system of claim 10, where the patient has at least one of: Thyroid, Celiac, Type 2 Diabetes, PCOS, or any combination thereof.
